(19) United States
(12) Patent Application Publication
(10) Pub. No.: US 2024/0025836 A1
(43) Pub. Date: Jan. 25, 2024

(54) FERULIC ACID GRANULES AND METHOD FOR THE PREPARATION THEREOF

(71) Applicant: RHODIA OPERATIONS, Lyon (FR)

(72) Inventors: Tiago Rafael Alves Mendes, Solaize (FR); Morad Assam, Chambost-Longessaigne (FR)

(73) Assignee: RHODIA OPERATIONS, Lyon (FR)

(21) Appl. No.: 18/256,533

(22) PCT Filed: Dec. 21, 2021
(86) PCT No.: PCT/EP2021/086948

§ 371 Date: Jun. 8, 2023

(30) Foreign Application Priority Data
Dec. 22, 2020 (FR) FR2013918

(51) Int. Cl. C07C 59/64; C07C 51/64; C12P 7/22

(52) U.S. Cl. CPC C07C 59/64; C07C 51/64; C12P 7/22

(57) ABSTRACT

The invention relates to ferulic acid granules and the production method thereof. The invention is suitable for use in particular in the fields of food, cosmetics and flavours.

### TECHNICAL FIELD

The present invention relates to ferulic acid granules and method for the preparation thereof. The invention has applications especially in the field of foodstuffs, cosmetics, and flavorings.

### PRIOR ART

Ferulic acid or 3-(4-hydroxy-3-methoxyphenyl)prop-2-enoic acid is an antioxidant naturally present in plants and especially cereals such as rice, maize, wheat or oats. It may also be present in solid or liquid coproducts of the food processing industry, in particular the oil production, cereal, sugar or alcohol sectors.

Ferulic acid can be prepared by chemical synthesis or by a biotechnological route involving a microbial fermentation or a plant tissue culture. It can also be obtained by a route described as natural and/or biobased in which a plant material is treated in order to extract ferulic acid from said plant material. For example, it can be extracted from by-products of the food processing industry or from seeds, for example according to the process described in WO2014/187784. Ferulic acid is used in various areas ranging from cosmetics to foodstuffs, particularly in the preparation of the flavoring substance vanillin.

Document WO2004/110975 describes the treatment of the liquor from the lime-cooking of maize kernels, which results in an effluent known as nejayote that contains ferulic acid, the treatment comprising a filtration, an acidification, the adsorption of the ferulic acid on a matrix, and then the washing of the matrix and elution with an organic solvent. The ferulic acid recovered is subsequently subjected to an additional step of recrystallization.

Document CN104628553 describes the purification of an alkaline solution of ferulic acid in which the solution is subjected to successive passes through membrane separation systems that can result in losses of ferulic acid and a decrease in the ferulic acid yield. The permeate subsequently obtained is acidified so as to precipitate the ferulic acid, which is recovered by centrifuging or filtration. The ferulic acid obtained is subjected to an additional step of purification over activated carbon and purification by recrystallization.

Document EP 3 612 511 describes the extraction and purification of ferulic acid from biomass of agricultural origin. The ferulic acid extracted may be purified by extraction methods employing an organic solvent.

The publication by H. Chen et al, in International Journal of Pharmaceuticals 574 (2020) 118914 describes various methods for the crystallization of ferulic acid with which it possible to obtain ferulic acid agglomerates. However, these methods require in particular the use of a binder so as to permit the agglomeration of the fine crystals formed. These methods permit the preparation of ferulic acid granules having a particle size represented by a median diameter of less than 400 μm. With the majority of such methods it is possible to obtain a ferulic acid powder. Drawbacks arising therewith are the presence of fines that cause problems with dust during the storage and handling of said powder. Such dusts are not without dangers associated with the risk of dust explosion, or risk to the health of personnel.

One of the objectives of the present invention is to provide a new presentation of ferulic acid and also a method of preparation with which it possible to overcome the abovementioned drawbacks.

### BRIEF DESCRIPTION OF THE INVENTION

The present invention relates to ferulic acid granules having a particle size expressed as a median diameter (d₅₀) of greater than or equal to 400 μm and a d₁₀ of between 150 μm and 250 μm.

The present invention relates also to trans-ferulic acid granules having a particle size expressed as a median diameter (d₅₀) of greater than or equal to 400 μm.

A third aspect of the present invention relates to a method for preparing ferulic acid granules by precipitation.

The present invention relates also to the use of ferulic acid according to the present invention in cosmetics, pharmaceuticals or foodstuffs.

Lastly, the present invention relates to a method for preparing vanillin by fermentation of ferulic acid according to the present invention.

### DESCRIPTION OF THE FIGURES

FIG. 1 shows a photograph taken with a Leica M420 microscope at 25× magnification of ferulic acid granules according to the present invention.

FIG. 2 is a photograph of ferulic acid (Leica M420 microscope at 23.9× magnification) according to example 2 (comparative).

FIG. 2 is a photograph of ferulic acid in powder form (Leica M420 microscope at 25× magnification).

### DETAILED DESCRIPTION

In the context of the present invention, and unless otherwise indicated, the expression “between . . . and . . . ” includes the limits. In the context of the present invention, unless otherwise indicated, the term “comprising . . . ” has the meaning of “consisting of . . . ”.

The present invention relates to ferulic acid granules having a particle size expressed as a median diameter (d₅₀) of greater than or equal to 400 μm and a d₁₀ of between 150 μm and 250 μm. The granules according to the present invention have particular physicochemical properties presented below.

According to a particular aspect, the ferulic acid according to the present invention may be of natural or biobased origin.

Ferulic acid of natural origin corresponds to the formula below, the double bond of ferulic acid of natural origin being in the trans position:

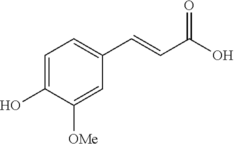 [SMILES: COc1cc(/C=C/C(=O)O)ccc1O]

Ferulic acid, when biobased, may be designated a “natural product”. According to the regulations in Europe and in the United States, this means that the product is obtained by physical, enzymatic or microbiological processes starting from plant or animal materials. Biobased ferulic acid is understood to mean ferulic acid entirely or significantly of plant or marine origin. For example, biobased ferulic acid may be obtained from agricultural by-products, plants, seeds, forestry materials or algae. In particular, biobased ferulic acid is of plant origin. Biobased ferulic acid thus does not result from a chemical synthesis.

The present invention relates also to trans-ferulic acid granules having a particle size expressed as a median diameter (d₅₀) of greater than or equal to 400 μm.

The ferulic acid granules according to the present invention are in the form of beads; these beads are essentially spherical in shape and the particle size distribution is preferably monomodal. The median diameter (d₅₀) of the ferulic acid granules is thus greater than or equal to 400 μm. The median diameter of the ferulic acid granules is generally greater than or equal to 500 μm, preferably greater than or equal to 550 μm, and very preferably greater than or equal to 600 μm. The median diameter of the ferulic acid granules is generally less than or equal to 2.0 mm, preferably less than or equal to 1.5 mm, and very preferably less than or equal to 1.0 mm. The median diameter of the ferulic acid granules may be less than or equal to 950 μm, preferably less than or equal to 900 μm, and very preferably less than or equal to 800 μm. The median diameter is defined as being when 50% by weight of the particles have a diameter greater or less than the median diameter.

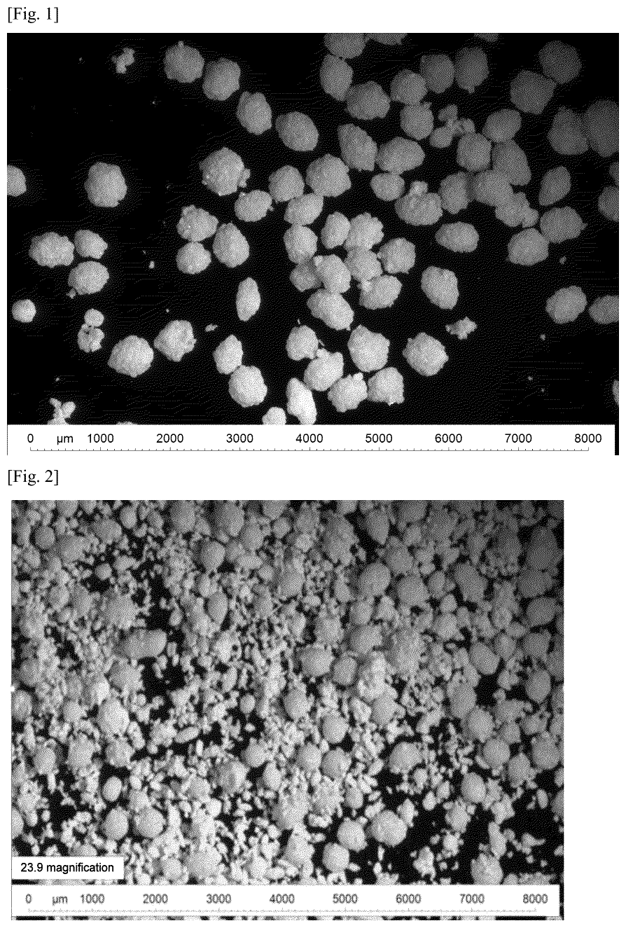

FIG. 1 depicts a photograph taken under an optical microscope that illustrates the bead-like morphology of the ferulic acid obtained according to the invention. A uniform particle size distribution is observed in the product obtained.

According to the present invention, the ferulic acid granules have a d₁₀ of between 150 μm and 250 μm. The d₁₀ is defined as being when 10% by weight of the particles have a diameter less than or equal to said d₁₀ value. By way of illustration, a d₁₀ of 200 μm means that 10% of the particles are less than or equal to 200 μm in size.

The ferulic acid granules according to the present invention have a percentage by volume of fine particles having a diameter of less than or equal to 150 μm that is less than or equal to 3.0%, preferably less than or equal to 2.0%.

The ferulic acid granules according to the present invention have a percentage by volume of fine particles having a diameter of less than or equal to 100 μm that is less than or equal to 2.0%, preferably less than or equal to 1.0%.

The ferulic acid has a minimum ignition energy of less than 3 mJ. The minimum ignition energy is measured as being the minimum energy that needs to be supplied to the mixture, in the form of a flame or a spark, in order to cause ignition. Thus, the very low content of fine particles (smaller than 150 μm and smaller than 100 μm) in the ferulic acid granules according to the present invention is particularly advantageous, reducing the risk of explosion. The ferulic acid granules according to the present invention thus improve the handling and storage conditions of ferulic acid compared to ferulic acid in powder form.

According to a particular aspect, the content of ferulic acid, preferably of trans-ferulic acid, in the ferulic acid granules according to the present invention is greater than or equal to 75% by weight, preferably greater than or equal to 80% by weight, very preferably greater than or equal to 90% by weight, even more preferably greater than or equal to 95% by weight, and very preferably greater than or equal to 99% by weight.

Advantageously, the granules according to the present invention have a physical form that gives them good resistance to abrasion, especially during transport and storage operations. These granules have good flowability properties, which is particularly advantageous during handling operations. These ferulic acid granules also have good dissolution properties, comparable in particular to those of ferulic acid in powder form. The granules can be used in the same applications as ferulic acid in powder form.

The ferulic acid granules according to the present invention may be used especially in a method for preparing vanillin, especially by fermentation. In particular, the yields of the fermentation method are not adversely affected by the use of said granules.

The present invention relates to a method for preparing ferulic acid granules by precipitation of a ferulic acid solution.

According to the present invention, the term “precipitation” refers to a process of conversion by chemical reaction of a chemical substance into a solid from a solution. The solid formed is the precipitate. A chemical agent is used to cause the formation of the precipitate.

According to the present invention, the method includes a step prior to the precipitation so as to permit the preparation of a ferulic acid solution. During this prior step, a ferulic acid powder undergoes dissolution. Preferably, the ferulic acid powder undergoes dissolution in aqueous solution.

According to a particular aspect, the content of ferulic acid, preferably of trans-ferulic acid, in the ferulic acid powder used is greater than or equal to 75% by weight, preferably greater than or equal to 80% by weight, very preferably greater than or equal to 82% by weight.

The pH of the ferulic acid solution obtained on conclusion of the prior step is greater than or equal to 7. The ferulic acid in solution may be in the form of a salt of ferulic acid, such as sodium ferulate or potassium ferulate in particular.

The prior step is generally carried out at a temperature of between 25° C. and 100° C., preferably between 40° C. and 60° C., even more preferably between 45° C. and 55° C., for example at 50° C. The concentration of ferulic acid or of the corresponding salt is usually greater than or equal to 2% by weight, preferably greater than or equal to 3% by weight, very preferably greater than or equal to 4% by weight. The concentration of ferulic acid or of the corresponding salt is generally less than or equal to 15% by weight relative to the total weight of the solution, preferably less than or equal to 13% by weight, very preferably less than or equal to 10% by weight.

The method of the present invention comprises a step of precipitating the ferulic acid solution obtained on conclusion of the prior step. The precipitation step is generally carried out by adding at least one mineral acid, preferably a very water-soluble mineral acid. Preferably at least one mineral acid is selected from acids having a pKa of less than or equal to 2, more preferably selected from the group consisting of hydrochloric acid, sulfuric acid, hydrobromic acid, perchloric acid, p-toluenesulfonic acid, nitric acid or chloric acid.

In the precipitation step, the at least one mineral acid is added to the ferulic acid solution so as to attain a pH of between 1 and 5, preferably between 2 and 3. The rate of addition of the mineral acid must generally be rapid. The at least one mineral acid is generally added at a rate greater than or equal to 1 mol[H⁺]/h/mol of initial ferulic acid. By way of example, when sulfuric acid is used, this is added at a rate that may be 0.5 mol[H₂SO₄]/hour/mol of initial ferulic acid. Operating at a lower rate was surprisingly found to result in reduced agglomeration to form ferulic acid granules. The granules obtained have a smaller particle size.

The precipitation step is generally carried out at a temperature of between 25° C. and 55° C. The temperature may be kept constant throughout the precipitation or may vary in the course of the precipitation. The precipitation step is preferably carried out at atmospheric pressure. The precipitation is usually carried out with stirring.

The method of the present invention permits the preparation of ferulic acid granules having the properties described above. In particular, the method of the present invention permits the preparation of ferulic acid granules in which the particle size represented by the median diameter is greater than or equal to 400 μm.

It is advantageous when the content of ferulic acid, preferably of trans-ferulic acid, in the ferulic acid granules according to the present invention is greater than or equal to 75% by weight, preferably greater than or equal to 80% by weight, very preferably greater than or equal to 82% by weight. The ferulic acid content in the ferulic acid granules is generally higher than the ferulic acid content in the ferulic acid powder used in the precipitation process. The precipitation process is thus also capable of achieving purification of the ferulic acid used.

The present invention relates also to granules of ferulic acid obtainable by the method according to the present invention.

The present invention relates to the use of ferulic acid according to the present invention or obtainable by the method according to the present invention in cosmetics, pharmaceuticals or foodstuffs. Advantageously, cosmetic, pharmaceutical or food products using these ferulic acid granules have properties comparable to products using ferulic acid powder.

Lastly, the present invention relates also to a method for preparing vanillin, in particular natural vanillin, by fermentation of ferulic acid according to the present invention or obtainable by the method according to the present invention.

### EXAMPLES

The examples below are intended to illustrate the invention without however limiting it.

### Example 1 (Inventive)

A 7.7% by weight solution of sodium ferulate is heated to 50° C. with stirring. The pH of the solution is about 7.

An aqueous sulfuric acid solution (32% by weight) is added at a rate of 1 mol[H⁺]/h/mol of initial ferulic acid so as to attain a pH of between 2 and 3.

The ferulic acid precipitates and the precipitate is filtered off at room temperature, washed with water and dried at 45° C.

The ferulic acid obtained has the characteristics shown in Table 1.

### Example 2 (Comparative)

A 7.7% by weight solution of sodium ferulate is heated to 50° C. with stirring. The pH of the solution is about 7.

An aqueous sulfuric acid solution (32% by weight) is added at a rate of 0.62 mol[H⁺]/h/mol of initial ferulic acid so as to attain a pH of between 2 and 3.

The ferulic acid precipitates and the precipitate is filtered off at room temperature, washed with water and dried at 45° C.

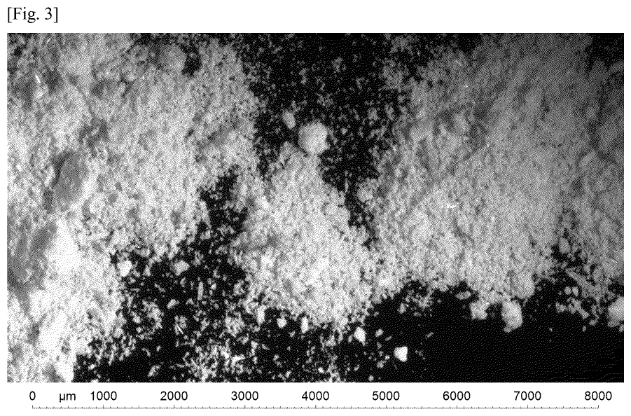

The ferulic acid obtained has the characteristics shown in Table 1.

| | Precipitated according | Precipitated according |
|---|---|---|
| | to example 1 | to example 2 |
| | (inventive) | (comparative) |
| Addition rate of the | 1 | 0.62 |
| sulfuric acid | | |
| (mol[H⁺]/h/mol of | | |
| initial ferulic acid) | | |
| d₅₀ (μm) | 680 | <390 |
| d₁₀ (μm) | 220 | Not measured |
| | FIG. 1 | FIG. 2 |

The ferulic acid obtained by the method of the invention has improved properties compared to ferulic acid powder (FIG. 3). The morphology of the ferulic acid according to the invention is very different to that of the powder or of the ferulic acid according to example 2, in particular only very few fine particles are observed. (FIGS. 1 to 3)

1. Ferulic acid granules having a particle size expressed as a median diameter of greater than or equal to 400 μm and having a d₁₀ of between 150 μm and 250 μm.

2. Ferulic acid granules as claimed in claim 1, wherein the percentage by volume of fine particles having a diameter of less than or equal to 100 μm is less than or equal to 2.0%.

3. Ferulic acid granules of natural origin having a particle size expressed as a median diameter of greater than or equal to 400 μm.

4. A method for preparing ferulic acid granules by precipitation of a ferulic acid solution.

5. The method for preparing ferulic acid granules as claimed in claim 4, wherein said precipitation is carried out by adding at least one mineral acid.

6. The method for preparing ferulic acid granules as claimed in claim 4, comprising a step prior to the precipitation of solubilizing of a ferulic acid powder,
wherein the ferulic acid powder undergoes dissolution so as to permit the preparation of a ferulic acid solution.

7. The method for preparing ferulic acid granules as claimed in claim 6, wherein the pH of the ferulic acid solution obtained on conclusion of the prior step is greater than or equal to 7.

8. The method as claimed in claim 6, wherein the prior step is carried out at a temperature of between 25° C. and 100° C.

9. The method for preparing ferulic acid granules as claimed in claim 4, wherein the at least one mineral acid is added at a rate greater than or equal to 1 mol[H⁺]/h/mol of initial ferulic acid.

10. The method for preparing ferulic acid granules as claimed claim 4, wherein the at least one mineral acid is added to the ferulic acid solution so as to attain a pH of between 1 and 5 in the ferulic acid solution.

11. A composition comprising ferulic acid granules as claimed in claim wherein the composition is used in cosmetics, pharmaceuticals or foodstuffs.

12. A method for preparing vanillin by fermentation of ferulic acid granules as claimed in claim 1.

13. The method of claim 5, wherein the mineral acid is selected from acids having a pKa of less than or equal to 2.

14. The method of claim 5, wherein the at least one mineral acid is selected from the group consisting of hydrochloric acid, sulfuric acid, hydrobromic acid, perchloric acid, p-toluenesulfonic acid, nitric acid, chloric acid, and combinations thereof.

15. The method of claim 6, wherein the ferulic acid powder undergoes dissolution in an aqueous solution.
